(19) United States
(12) Patent Application Publication
(10) Pub. No.: US 2007/0250007 A1
(43) Pub. Date: Oct. 25, 2007

(54) Drug Delivery Device With Air Pressure Spring And Safety Valve

(75) Inventor: Avraham Shekalim, Nesher (IL)

Correspondence Address: DR. MARK M. FRIEDMAN;C/O BILL POLKINGHORN - DISCOVERY DISPATCH, 9003 FLORIN WAY, UPPER MARLBORO, MD, 20772, US

(73) Assignee: NILIMEDIX LTD., Haifa (IL)

(21) Appl. No.: 11/738,533

(22) Filed: Apr. 23, 2007

Related U.S. Application Data
(60) Provisional application No. 60/745,406, filed on Apr. 23, 2006.

(51) Int. Cl. A61M 37/00

(52) U.S. Cl. 604/131

(57) ABSTRACT

A drug delivery device includes a drug reservoir which is pressurized by the pressure of air compressed during the course of insertion of a reservoir container itself. Also provided is a safety cut-off valve arrangement in which a two-valve flow control system is further supplemented by a safety cut-off valve which closes the flow path whenever the electronics unit required for operation of the device is not properly installed.

### FIELD AND BACKGROUND OF THE INVENTION

The present invention relates to drug delivery systems and, in particular, it concerns body-mounted pumps for infusion of low dosage drugs, such as are used for insulin administration, More specifically, the invention relates to an infusion pump in which a drug reservoir is pressurized by the pressure of air compressed during the course of insertion of a reservoir container itself. In another aspect, the invention relates to a failsafe valve arrangement in which a two-valve normally closed flow control system is further supplemented by a failsafe valve which closes the flow path whenever the electronics unit required for operation of the device is not properly installed.

The present invention is based upon principles of implementation as described in the following patents and patent applications: U.S. Pat. No. 6,736,796; PCT Patent Application Publication No. WO03/045302; U.S. Patent Application Publication No. US 2003-0216683; and PCT Patent Application Publication No. WO2004/105827. All of the above-mentioned publications are hereby incorporated by reference in their entirety. Unless and except where disclosed otherwise, it should be assumed that the structure and operation of the present invention are as described in the above-referenced documents, and may be fully understood by reference thereto.

Thus, in accordance with the aforementioned publications, the present invention relates to a medication delivery pump which employs a pressurized reservoir for delivering a drug through a control system of two valves and a flow restriction, all under the control of an electronic system which receives inputs from a pressure sensor detecting pressure at, or differential pressure between, two points on the flow path.

There is a need for simple and effective ways of pressurizing a fluid drug within a reservoir of the device without unduly adding to the weight and complexity of the device. I would also be advantageous to provide a safety cut-off valve which would serve as an additional precaution that no drug be released from the device if the electronic control unit is not properly engaged with the control valve of the flow arrangement.

### SUMMARY OF THE INVENTION

The present invention is an infusion pump in which a drug reservoir is pressurized by the pressure of air compressed during the course of insertion of a reservoir container itself. According to another aspect, the invention provides a safety cut-off valve arrangement in which a two-valve flow control system is further supplemented by a safety cut-off valve which closes the flow path whenever the electronics unit required for operation of the device is not properly installed.

According to the teachings of the present invention there is provided, a drug delivery system comprising: (a) a housing containing a pressure chamber including a parallel-sided shaft terminating at an open end; (b) a drug cartridge having an internal storage volume containing a quantity of a liquid drug, the drug cartridge being configured to be inserted along at least part of the parallel-sided shaft, the drug cartridge including a seal arrangement deployed for sealing between the drug cartridge and the shaft such that insertion of the drug cartridge into the shaft traps a quantity of gas at ambient pressure within the pressure chamber and compresses the quantity of gas to a raised pressure above ambient pressure, wherein the drug cartridge is formed with a pressure transfer opening arranged to expose the internal storage volume to the raised pressure, thereby pressurizing the internal storage volume.

According to a further feature of the present invention, the internal storage volume is configured as a parallel-sided vessel, and wherein a piston is interposed in sliding engagement with the vessel between the liquid drug and the pressure transfer opening.

According to a further feature of the present invention, the drug cartridge is configured such that, after sliding of the piston to a position corresponding to release of all of the liquid drug, the raised pressure is at least about 30 percent above the ambient pressure.

According to a further feature of the present invention, the shaft is substantially cylindrical, and wherein the parallel-sided vessel is substantially cylindrical.

According to a further feature of the present invention, the drug cartridge is formed with an elastomeric seal, the drug delivery system further comprising a cartridge interface including a needle deployed for piercing the elastomeric seal.

According to a further feature of the present invention, the cartridge interface further includes a retaining configuration for retaining the drug cartridge in an inserted state so as to maintain the raised pressure.

There is also provided according to the teachings of the present invention, a drug delivery system comprising: (a) a disposable unit including: (i) a drug reservoir, (ii) a fluid outlet, and (iii) a fluid flow arrangement defining a flow path from the drug reservoir to the fluid outlet, the fluid flow arrangement including two fluid flow control valves and a fluid flow restriction; and (b) a control unit configured for mating with the disposable unit, the control unit including: (i) an electronic control system, and (ii) an actuator arrangement deployed to cooperate with the two fluid flow control valves to selectively actuate the fluid flow control valves, wherein the fluid flow arrangement further includes a normally-closed cut-off valve configured to interrupt the flow path and selectively opened by mechanical engagement with a part of the control unit when the control unit is mated with the disposable unit.

### BRIEF DESCRIPTION OF THE DRAWINGS

The invention is herein described, by way of example only, with reference to the accompanying drawings, wherein:

FIG. 1 is an overview of the main functional elements of the drug delivery device upon which the present invention is based;

FIGS. 2A and 2B are overall isometric views of the drug delivery device of the present invention;

FIG. 3 is a cross-sectional view taken through the drug delivery device of FIG. 2A;

FIG. 4 is a partially exploded view of a disposable unit from the drug delivery device of FIG. 2A;

FIG. 5 is an isometric view of a drug cartridge and cartridge interface from the drug delivery device of FIG. 2A;

FIGS. 6A and 6B are isometric views during insertion of the drug cartridge of FIG. 5 into the disposable unit of FIG. 4;

FIGS. 7A and 7B are schematic cross-sectional views at two stages during insertion of the drug cartridge of FIG. 5 into the disposable unit of FIG. 4;

FIG. 8 is an isometric view taken during engagement of a control unit from the drug delivery device of FIG. 2A with the disposable unit;

FIG. 9A is a partial cross-sectional view showing the engagement of the control unit with a safety cut-off valve according to a second aspect of the present invention;

FIG. 9B is a schematic cross sectional view taken through the cut-off valve itself and

FIG. 10 is an isometric view of the drug delivery device of FIG. 2A ready for use.

### DESCRIPTION OF THE PREFERRED EMBODIMENTS

The present invention is an infusion pump in which a drug reservoir is pressurized by the pressure of air compressed during the course of insertion of a reservoir container itself. According to another aspect, the invention provides a safety cut-off valve arrangement in which a two-valve flow control system is further supplemented by a safety cut-off valve which closes the flow path whenever the electronics unit required for operation of the device is not properly installed.

The principles and operation of drug delivery devices according to the present invention may be better understood with reference to the drawings and the accompanying description.

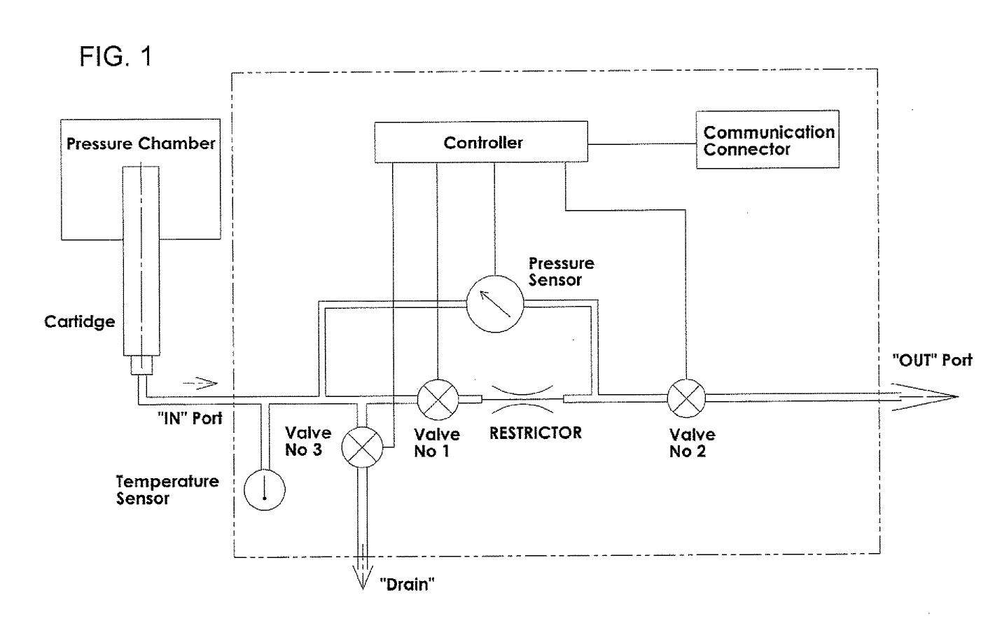

Referring now to the drawings, FIG. 1 gives an overview of the function of the device of the present invention according to a preferred embodiment modeled on the teachings of the aforementioned publication US 2003-0216683. Specifically, the drug delivery device shown includes a drug reservoir, a fluid outlet, and a fluid flow arrangement defining a flow path from the drug reservoir to the fluid outlet. The fluid flow arrangement includes in series a first fluid flow control valve, a fluid flow restriction and a second fluid flow control valve. A pressure sensor, in this case a differential pressure sensor, optionally supplemented by a temperature sensor, provides information from which flow rates and various self-testing functions may be performed, all as detailed in the aforementioned reference. In the particularly preferred embodiment shown here, a third valve provides an emergency release function, discharging the reservoir pressure in the event that a critical fault occurs to prevent delivery of excess quantities of the drug to the user. The various sensors are connected to an electronic control system which selectively operates the various valves via an actuator arrangement. The outlet is typically connected, either directly or indirectly, to an infusion set for delivering the drug into the body. In this case, the infusion set is shown as an integrated infusion set provided at the periphery of the drug delivery device.

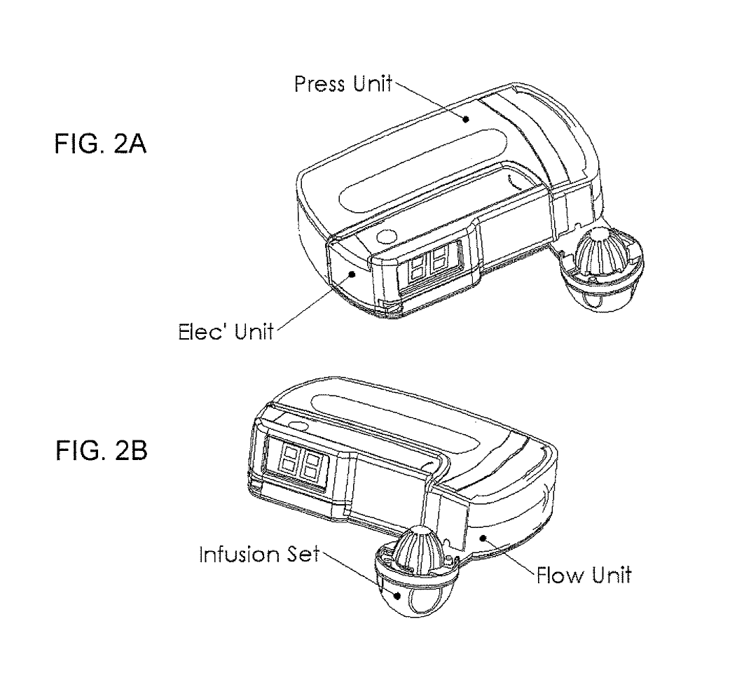

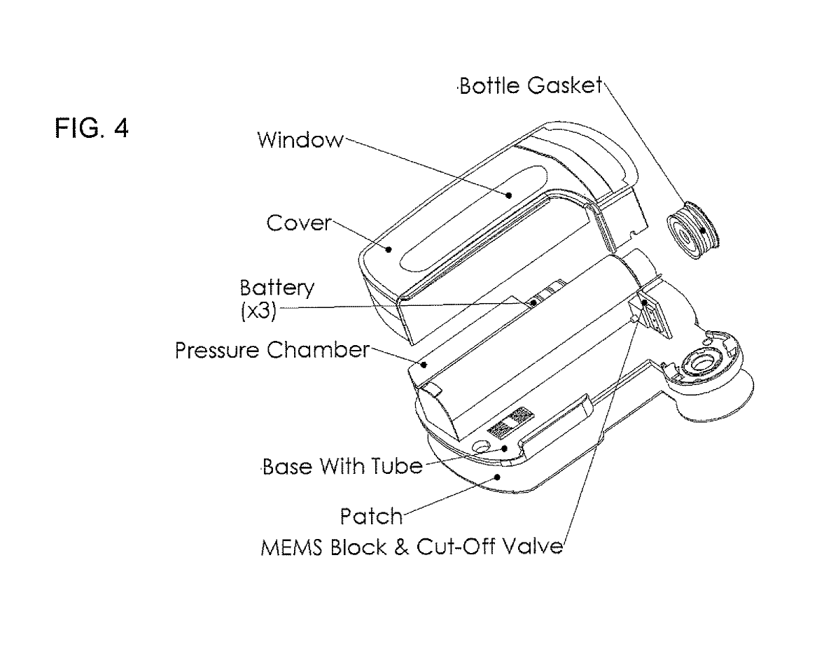

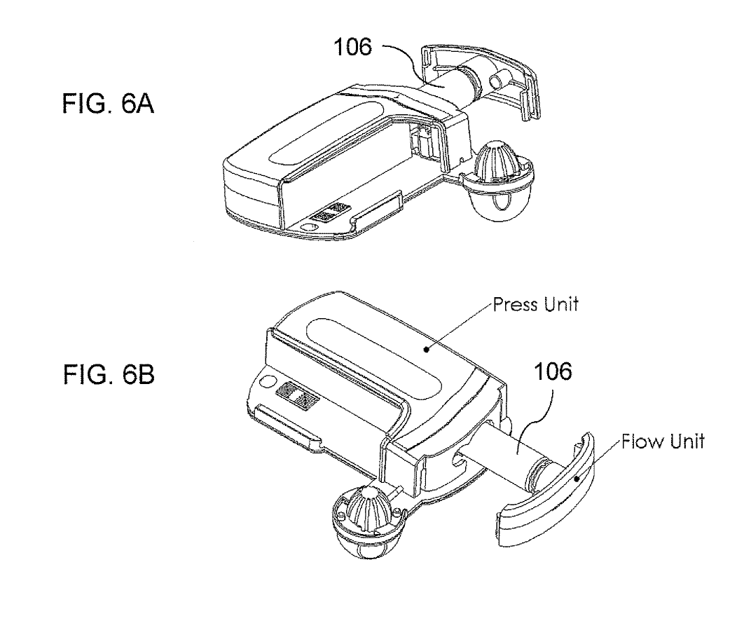

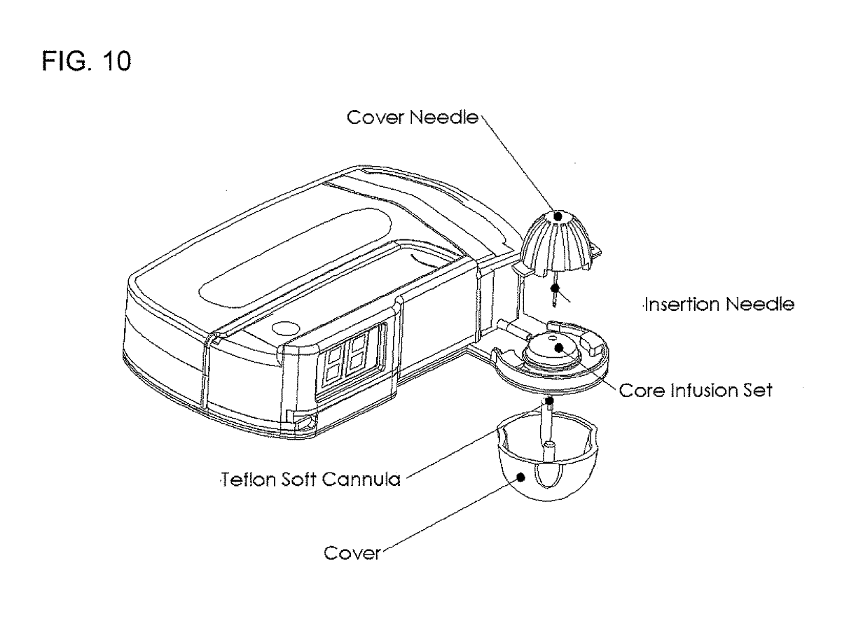

The remaining FIGS. 2A-10 show various aspects of a preferred implementation of the device of FIG. 1, illustrating a number of inventive aspects of the present invention. Of particular importance are two aspects, each of which is believed to be patentable in its own right. The first is an air spring arrangement for pressurizing the drug reservoir. The second is a safety shut-off valve. These two aspects will now be addressed individually.

### Air-Spring Primed By Insertion Of A Drug Reservoir Bottle

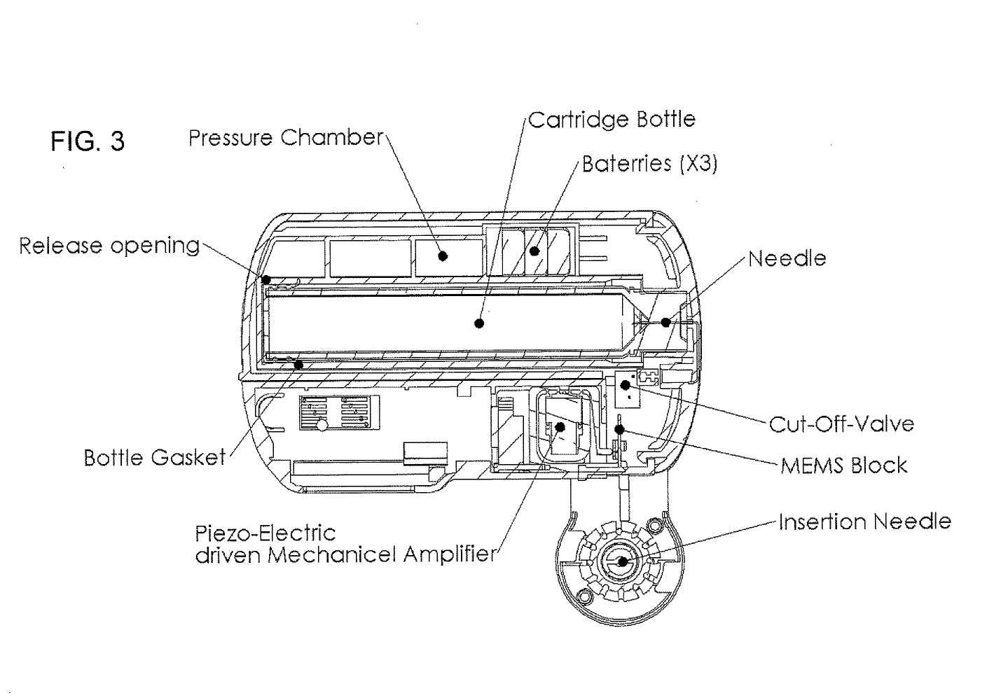

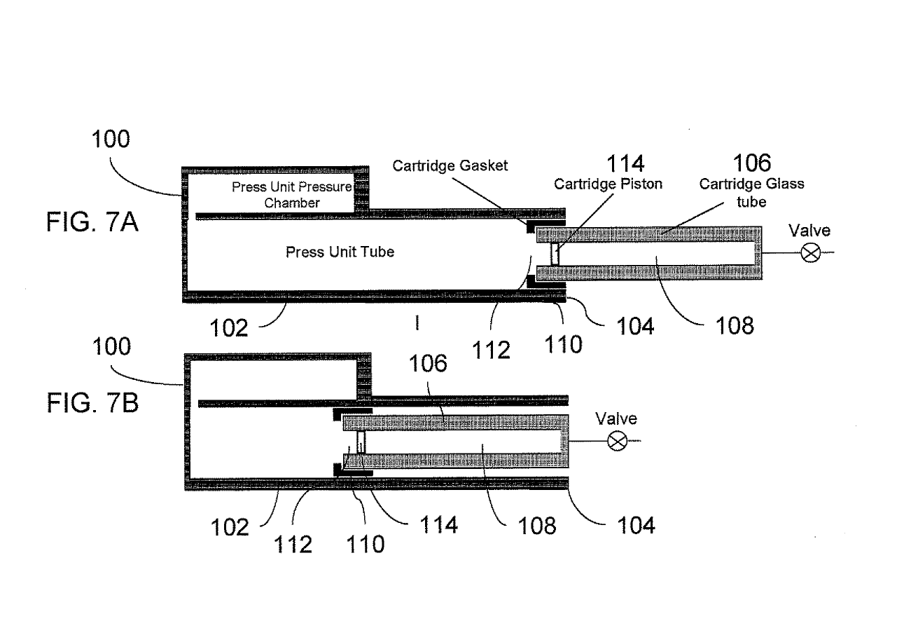

Referring particularly to FIGS. 3-7B, there is illustrated a structure and method for insertion of a drug reservoir bottle into the disposable drug delivery device. As best seen in FIGS. 7A and 7B, it is a particularly preferred feature of the present invention that an elevated pressure in the drug delivery reservoir is generated primarily, and preferably exclusively, by compression of air during insertion of the reservoir bottle. The use of an air spring in this manner provides the required delivery pressure for delivering the drug to the user, while avoiding any addition to the weight of the unit by mechanical springs or other biasing elements.

Although the driving pressure is generated only by insertion of the reservoir bottle, it should be noted that preferred implementations of the invention ensure sufficient residual pressure to drive drug delivery until the liquid drug content is exhausted. This residual pressure when the bottle is empty results from the extra air displacement due to the thickness of the walls of the bottle itself and/or from extra volume outside the bottle swept through by the cartridge gasket which effectively acts as a compression piston in sealing contact with the wall of the pressure chamber.

Thus, there is shown a drug delivery system having a housing containing a pressure chamber 100 including a parallel-sided shaft 102 terminating at an open end 104. A drug cartridge 106, having an internal storage volume 108 containing a quantity of a liquid drug, is configured to be inserted along at least part of the parallel-sided shaft. A seal arrangement (gasket 110) is deployed to seal between drug cartridge 106 and shaft 102 such that insertion of the drug cartridge into the shaft traps a quantity of gas at ambient pressure within the pressure chamber and compresses the quantity of gas to a raised pressure above ambient pressure. The drug cartridge is formed with a pressure transfer opening 112 arranged to expose the internal storage volume to the raised pressure, thereby pressurizing the internal storage volume.

Preferably, the internal storage volume 108 is configured as a parallel-sided vessel, and a piston 114 is interposed in sliding engagement with the vessel between the liquid drug and the pressure transfer opening. The thickness of the walls of cartridge 106 and/or the volume swept through by gasket 110 are preferably chosen such that, after piston 114 has been displaced to a position corresponding to release of all of the liquid drug, the raised pressure is at least about 30 percent above the ambient pressure. In other words, the volume of the pressure chamber is preferably reduced by the presence of gasket 110 and cartridge 106 by at least about 25%, and more preferably by at least about 50%, even after the liquid contents have been dispensed. In order to avoid excessive compression, and corresponding excessive resistance to insertion of the drug reservoir, the pressure chamber preferably has an excess volume beyond the volume swept out by cartridge 106. In the embodiment illustrated here, the excess volume is located at least in part in an interconnected side chamber lying outside shaft 102.

For particular simplicity of implementation of the air-tight seals, both shaft 102 and parallel-sided vessel 108 are implemented with substantially cylindrical wall surfaces.

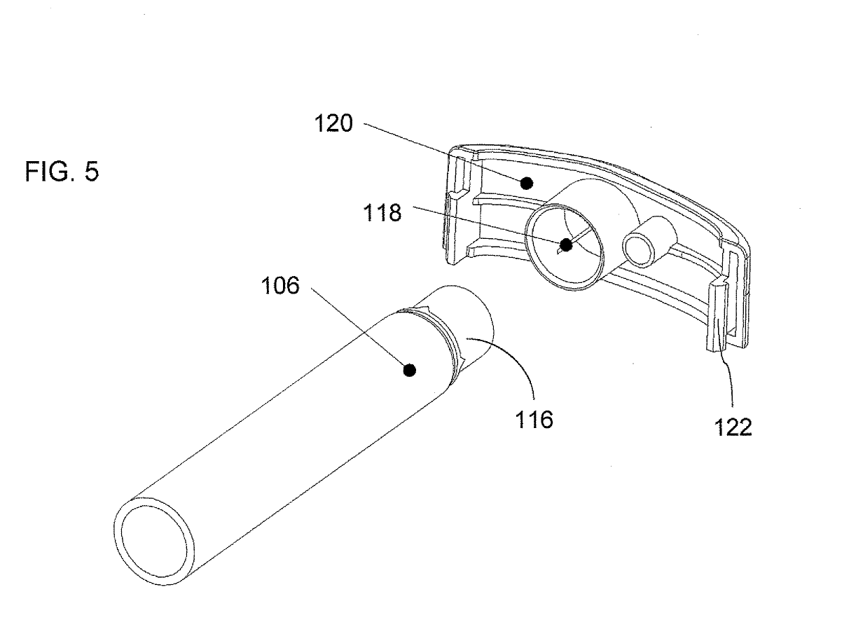

Connection of drug cartridge 106 for dispensing the drug is preferably performed via an elastomeric seal 116 which is pierced by a needle 118 formed as part of a cartridge interface 120 (see FIG. 5). Needle 118 conveys the drug to the fluid flow arrangement (not shown). Cartridge interface 120 preferably also includes a retaining configuration 122 for retaining the drug cartridge in an inserted state, thereby maintaining the raised pressure.

Optionally, cartridge 106 may be transparent, and a corresponding window may be provided in the housing to facilitate visual monitoring by a user of the remaining quantity of the drug.

### Safely Valve

Turning now to the second aspect of particular importance, as discussed in the aforementioned prior art references, the components of the drug delivery device are preferably subdivided between a disposable unit which includes the drug reservoir, the outlet and all components of the fluid flow arrangement which come into contact with the fluid, optionally as well other elements which need frequent replacement such as batteries. Other components of the device, and particularly an electronic control system and an actuator arrangement deployed to cooperate with and selectively actuate the fluid flow control valves, are included in a reusable control unit which is configured for mating with the disposable unit.

The aforementioned fluid flow control scheme employing two valves in series within the main flow path, and particularly including a third valve for emergency pressure release in the event of a critical malfunction, provides a high level of user safety. Nevertheless, due to the subdivision of components between two units, there may remain a concern of improper operation in the case that the reusable control unit is not properly engaged with the disposable unit. To address this risk, another aspect of the present invention provides a normally-closed cut-off valve integrated into the fluid flow arrangement and configured to interrupt the flow path. The normally-closed cut-off valve is selectively opened by mechanical engagement with a part of the control unit when the control unit is mated with the disposable unit.

Thus, the safety cut-off valve blocks passage of the liquid drug whenever the electronics unit is not fully inserted into the disposable pump unit. This additional safety cut-off valve is preferably used in all embodiments, but is of extra importance in implementations where the other valves of the flow path control system tend to an open state until pressure is applied to them by insertion of the electronics unit.

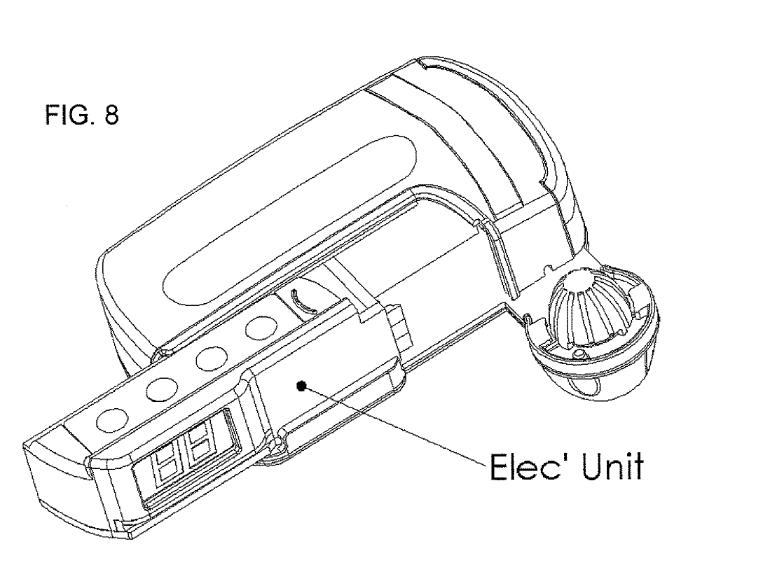

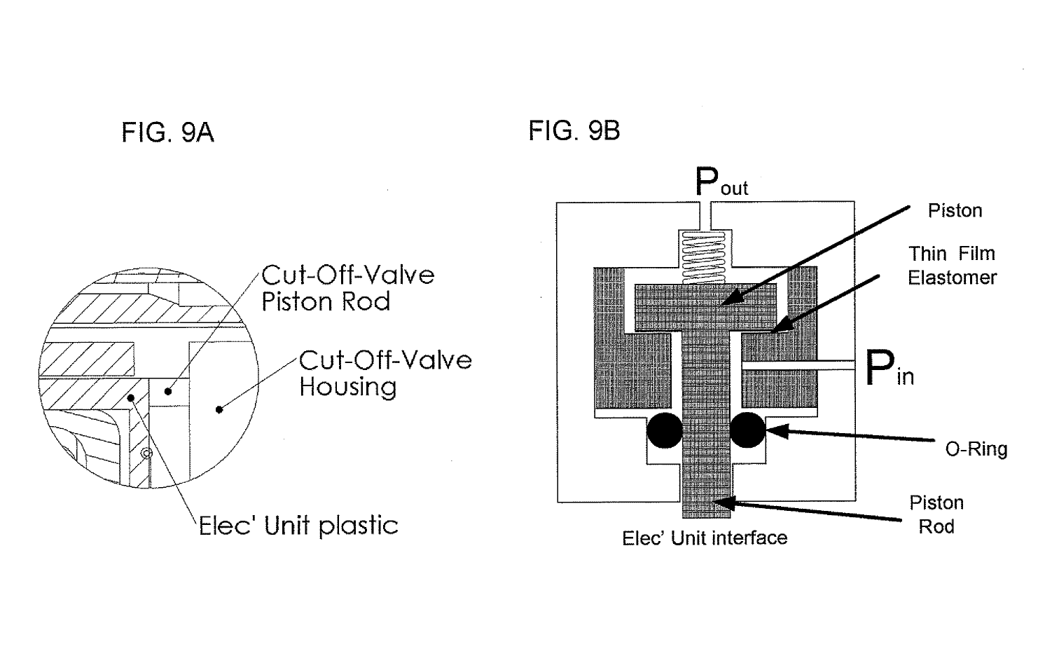

This aspect of the present invention is best understood with reference to FIGS. 8, 9A and 9B. Specifically, FIG. 8 shows a general view of the control unit (“electrical unit”) being mated with the disposable unit. FIG. 9A shows a magnified partial cross-sectional view of a corner of the electrical unit when fully inserted bearing on a piston rod of the cut-off valve. FIG. 9B shows schematically an exemplary implementation of the cut-off valve itself.

Referring not to the structure of the cut-off valve as illustrated particularly in FIG. 9B, when the Elec' unit isn't in “Lock In Position” the valve spring pushes the piston towards the a thin film elastomer seal and the drug flow is blocked. When the user slides the Elec' unit in fully (see FIG. 9A), it pushes the valve piston and the drug can freely flow around the piston and out thorough the hole under the spring. If for some reason the Elec' unit is disconnected from the disposable unit the cut-off valve is automatically closed and prevents the drug flow.

It will be appreciated that the above descriptions are intended only to serve as examples, and that many other embodiments are possible within the scope of the present invention as defined in the appended claims.

What is claimed is:

1. A drug delivery system comprising:
(a) a housing containing a pressure chamber including a parallel-sided shaft terminating at an open end; and
(b) a drug cartridge having an internal storage volume containing a quantity of a liquid drug, the drug cartridge being configured to be inserted along at least part of said parallel-sided shaft, said drug cartridge including a seal arrangement deployed for sealing between said drug cartridge and said shaft such that insertion of said drug cartridge into said shaft traps a quantity of gas at ambient pressure within said pressure chamber and compresses said quantity of gas to a raised pressure above ambient pressure,
wherein said drug cartridge is formed with a pressure transfer opening arranged to expose said internal storage volume to said raised pressure, thereby pressurizing said internal storage volume.

2. The drug delivery system of claim 1, wherein said internal storage volume is configured as a parallel-sided vessel, and wherein a piston is interposed in sliding engagement with said vessel between the liquid drug and said pressure transfer opening.

3. The drug delivery system of claim 2, wherein said drug cartridge is configured such that, after sliding of said piston to a position corresponding to release of all of the liquid drug, said raised pressure is at least about 30 percent above said ambient pressure.

4. The drug delivery system of claim 2, wherein said shaft is substantially cylindrical, and wherein said parallel-sided vessel is substantially cylindrical.

5. The drug delivery system of claim 1, wherein said drug cartridge is formed with an elastomeric seal, the drug delivery system further comprising a cartridge interface including a needle deployed for piercing said elastomeric seal.

6. The drug delivery system of claim 5, wherein said cartridge interface further includes a retaining configuration for retaining said drug cartridge in an inserted state so as to maintain said raised pressure.

7. A drug delivery system comprising:
(a) a disposable unit including:
(i) a drug reservoir,
(ii) a fluid outlet, and
(iii) a fluid flow arrangement defining a flow path from said drug reservoir to said fluid outlet, said fluid flow arrangement including two fluid flow control valves and a fluid flow restriction; and
(b) a control unit configured for mating with said disposable unit, said control unit including:
(i) an electronic control system, and
(ii) an actuator arrangement deployed to cooperate with said two fluid flow control valves to selectively actuate said fluid flow control valves,
wherein said fluid flow arrangement further includes a normally-closed cut-off valve configured to interrupt said flow path and selectively opened by mechanical engagement with a part of said control unit when said control unit is mated with said disposable unit.
